(19) United States
(12) Patent Application Publication
(10) Pub. No.: US 2013/0341223 A1
(43) Pub. Date: Dec. 26, 2013

(54) DISPOSABLE COVER FOR STETHOSCOPE

(71) Applicant: Avossi, Inc., Brooklyn, NY (US)

(72) Inventor: Peter Fong, Brooklyn, NY (US)

(21) Appl. No.: 13/917,804

(22) Filed: Jun. 14, 2013

Related U.S. Application Data
(60) Provisional application No. 61/659,518, filed on Jun. 14, 2012.

(51) Int. Cl. A61B 7/02

(52) U.S. Cl. CPC A61B 7/02
USPC 206/363

(57) ABSTRACT

A stethoscope cover having an enclosure with a closed end and an open end. An arrangement (e.g., a circular element) can be located substantially near the closed end can hold a bell of the stethoscope. A V-shaped elastic arrangement can be located at the open end and can extend the enclosure when earpieces of a stethoscope can be pulled apart. A strap can be located at the open and can hold the stethoscope in the enclosure.

### CROSS-REFERENCE TO RELATED APPLICATIONS

This application relates to and claims priority from U.S. Patent Application No. 61/659,518, filed on Jun. 14, 2012, the entire disclosure of which is incorporated herein by reference.

### FIELD OF THE DISCLOSURE

The present disclosure relates generally to a cover for a stethoscope, and more specifically, to exemplary embodiments of a disposable cover for use on, for example, a stethoscope.

### BACKGROUND INFORMATION

Primary-care physicians in the United States can examine, on average, 30 patients per day. Currently, specialty-care physicians can examine around 12-15 patients per day. During a physical examination, it can be a common practice to use a stethoscope to examine the patient. A stethoscope is a medical device used for auscultation, or listening to the internal sounds of a human body. This device can often be used to listen to lung and heart sounds. It can also be used to listen to intestines and blood flow in arteries and veins. During a physical examination, it can be common practice for physicians to wear disposable gloves, and to change the gloves between patients. Despite this safety procedure, it can be common practice that the same stethoscope be used on all of patients, and it can be uncommon for the stethoscope to be cleaned between examinations. Even if the stethoscope is cleaned after an examination, the stethoscope can still harbor residual microorganisms. This can lead to the transmission of an infection from one patient to another patient, or between the patient and the doctor. Most typically, when a stethoscope can be cleaned or covered, it may only be done so for the diaphragm. This can be insufficient for stopping cross contamination. For example, there may not be any protection for when a doctor examines a patient and the tube brushes against the infected patient. Additionally, even if the diaphragm can be covered or disinfected, the tube can be contaminated for the next patient. The earpieces of the stethoscope can also be a source of infection as a doctor removes the stethoscope after touching an infected patient during an exam. The next time the doctor puts the stethoscope on and touches the earpieces, his newly clean gloves can be contaminated for his or her next patient. In this manner, the entire stethoscope, including the earpieces, can be a risk for infection.

Studies have been performed that measure the levels of contamination of stethoscopes in various clinical settings including intensive care units, medicine wards and private clinics. In these studies, it was determined that stethoscopes can be a potential source of nosocomial infection (e.g., infections acquired in a hospital). During such studies, sampling of stethoscopes for bacterial cultures revealed that almost all stethoscopes can harbor microorganisms, and a significant number can contain antibiotic-resistant strains of bacteria (e.g., methicillin resistant Staph Aureus). Further, more than just the diaphragms can be contaminated. For example, a study of 56 patients was performed at Geneva University in which stethoscope contamination was measured after each patient's stethoscope examination. The study found that both the diaphragm and the tube can be significant sources of infection, and that there can be an insignificant difference in the level of contamination between the two areas.

Currently, the most common way to clean a stethoscope can be to swab the stethoscope with alcohol. Swabbing the stethoscope with alcohol has been shown to decrease the bacterial colonization of stethoscopes. However, health care providers, especially medical staff, do not regularly wipe their stethoscopes with alcohol. Even if the stethoscope diaphragm can be cleaned with alcohol, earpieces can continue to harbor organisms, and cleaning these can require much more time. Further, swabbing with alcohol leaves the stethoscope wet and cold and can cause discomfort to the patient.

To overcome the some of the above problems, single use disposable stethoscopes have been employed, which can be discarded after each use. Single use stethoscopes, however, can be very expensive, and generally have very poor sound quality.

Thus, it may be beneficial to provide a disposable cover for a stethoscope that can protect both the patient and the doctor from the transmission of infectious organisms or diseases, that is easily used, relatively inexpensive to manufacture, and which can overcome at least some of the deficiencies described herein above.

### SUMMARY OF EXEMPLARY EMBODIMENTS

Exemplary embodiments of the present disclosure relate to a disposable stethoscope cover. The exemplary stethoscope cover can provide a disposable protective cover for a stethoscope, which can be easily and quickly applied to a stethoscope to cover, for example, the diaphragm, bell, tube and a portion of the ear-piece of a stethoscope, and can help in preventing patient-to-patient and patient-to-health care provider transmission of infection by forming a barrier between the patient and/or doctor and the stethoscope.

The exemplary disposable stethoscope cover can be made from two layers of non-woven fabric, which can be stitched at the edges to form a pouch that can be used to cover the stethoscope. The exemplary disposable stethoscope cover can be open at the proximal end and closed at the distal end. A V-shape can be cut out of the two layers of fabric at the proximal end and triangular pieces of elastic fabric can be stitched to the edges of the V-shape such that when the elastic pieces can be stretched, the exemplary disposable stethoscope cover can easily be pulled over the earpieces of the stethoscope.

This exemplary disposable stethoscope cover can be easily fastened and/or removed, and can be applied on a stethoscope in few steps. The exemplary disposable stethoscope cover can then be discarded after a single use.

This exemplary disposable stethoscope cover, unlike disposable stethoscopes which can provide poor sound quality, does not sacrifice the sound quality of the stethoscope as the physicians can use their own stethoscopes, and the non-woven material can transmit sound perfectly.

According to an exemplary embodiment of the present disclosure, a stethoscope cover can include an enclosure having a closed end and an open end. A first arrangement can be located substantially near the closed end and can be configured to hold a bell of a stethoscope. An elastic second arrangement can be located at the open end and configured to extend the enclosure when earpieces of the stethoscope are pulled apart. The elastic arrangement can be substantially v-shaped. A can strap configured to hold the stethoscope in the enclosure, and the stethoscope cover can be made from a first material and the strap can be made from a second material that can be different from the first material. The strap can include a closure portion having a semi-permanent adhesive to attach one end of the strap to the stethoscope cover. The strap can include a closure portion including a hook and loop system to attach one end of the strap to the stethoscope cover.

In some exemplary embodiments of the present disclosure, the first arrangement can include a circular element, and can contain an antimicrobial agent. The stethoscope cover can be made from a first material and the first arrangement can be made from a second material that can be different from the first material. The first arrangement can be made from a non-porous material, and can extend a particular distance away from a surface of the stethoscope cover.

In certain exemplary embodiments of the present disclosure, the first arrangement can include at least two discs layered one on top of the other, which can contain an anti-microbial agent or a drug. The stethoscope cover can be sized to substantially cover an entirety of the stethoscope, or it can be sized to substantially cover an entirety of the stethoscope except for earpieces of the stethoscope. The stethoscope cover can be of unitary construction, or it can be constructed from a top layer stitched to a bottom layer. The first arrangement can be located on the top layer, and the elastic second arrangement can include two elastic portions, one elastic portion on the top layer and one elastic portion on the bottom layer.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is a perspective view of an exemplary disposable stethoscope cover with a stethoscope placed within, according to an exemplary embodiment of the present disclosure;

FIG. 2 is a perspective view of the exemplary disposable stethoscope cover according to FIG. 1 according to an exemplary embodiment of the present disclosure;

FIG. 3 is a close-up perspective view of one end of the exemplary disposable stethoscope cover of FIG. 1 according to an exemplary embodiment of the present disclosure; and

FIG. 4 is an enlarged perspective view of one end of the exemplary disposable stethoscope cover of FIG. 1 according to an exemplary embodiment of the present disclosure;

Throughout the drawings, the same reference numerals and characters, unless otherwise stated, are used to denote like features, elements, components, or portions of the illustrated embodiments. Moreover, while the present disclosure will now be described in detail with reference to the figures, it is done so in connection with the illustrative embodiments and is not limited by the particular embodiments illustrated in the figures.

### DETAILED DESCRIPTION OF EXEMPLARY EMBODIMENTS

The exemplary embodiments of the present disclosure may be further understood with reference to the following description and the related appended drawings. The exemplary embodiments of the present disclosure relate to a disposable cover for a stethoscope. For example, the exemplary disposable stethoscope cover can be configured to hold a stethoscope and protect a patient and a doctor from the transmission of diseases that can reside on the stethoscope. The exemplary embodiments are described with reference to a stethoscope, although those having ordinary skill in the art will understand that the exemplary embodiments of the present disclosure can be implemented on other medical or non-medical devices that can benefit from a disposable protective cover.

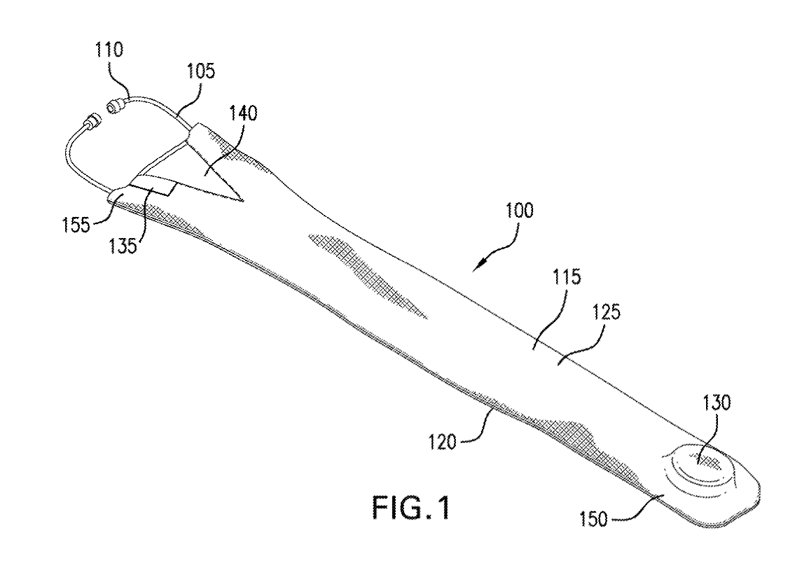

FIG. 1 shows a perspective view of an exemplary disposable stethoscope cover 100 according to an exemplary embodiment of the present disclosure. For example, the exemplary disposable stethoscope cover 100 can be long and slender, and can be designed to fit all or part of a stethoscope 105. As illustrated in FIG. 1, the exemplary disposable stethoscope cover 100 can cover all of stethoscope 105, except for, for example, the very top of earpieces 110, facilitating the user of stethoscope 100 to insert earpieces 110 into their ears while the exemplary disposable stethoscope cover 100 can be in use. Alternatively, the exemplary disposable stethoscope cover can cover earpieces 110, providing an additional barrier between the wearer of stethoscope 100 and stethoscope 100.

The exemplary disposable stethoscope cover 100 can be made from once piece of material, which can be connected, or stitched together at one end. Alternatively, the exemplary disposable stethoscope cover 100 can be made from multiple pieces of material stitched together. The exemplary disposable stethoscope cover 100 can have a pouch 115 made from a bottom layer 120 and a top layer 125, which together can form a pouch or open area into which stethoscope 105 can be inserted into. The pouch 115 can be made from a non-woven fabric, or from any fabric or material that can hold stethoscope 105.

The exemplary disposable stethoscope cover can have a closed end 150 and an open end 155 into which stethoscope 105 can be inserted. Closed end 150 can have a circular area 130 that can be designed to engage or hold the bell (not shown) of stethoscope 105. When stethoscope 105 can be inserted in the exemplary disposable stethoscope cover 100, the bell of stethoscope 105 can naturally fit into circular area 130.

Circular area 130 can be made from the same material as pouch 115, and can extend a distance away from the surface of pouch 115 such that circular area 130 may not be flush with pouch 115. Alternatively, circular area 130 can be flush with pouch 115. Additionally, circular area 130 can be made from a different material than that of pouch 115. According to one exemplary embodiment, circular area 130 can be two discs of non-woven fabric or a rubber polymer attached to pouch 115 such that they lie over the bell and diaphragm of stethoscope 105. The two discs can be impregnated or treated with an antimicrobial agent, or can have drug delivery polymer formulations that deliver antimicrobial agents, to aid in preventing the transmission of diseases.

According to another exemplary embodiment, circular area 130 can be made of any material, and can be of any design, such that it aids in preventing the transmission of diseases. For example, circular area 130 can be made from a non-porous material that may not facilitate the transmission of diseases. In addition, pouch 115 can be made from, or have a non-porous material woven inside, which may not facilitate for the transmission of diseases.

The exemplary disposable stethoscope cover 100 can have a strap 135, which can assist in holding stethoscope 105 inside of pouch 115. Additionally, the exemplary disposable stethoscope cover 100 can have a cutout 140, which can be elastic, to facilitate the user of stethoscope 105 to extend earpieces 110, in turn extending pouch 115, around their head and into their ears.

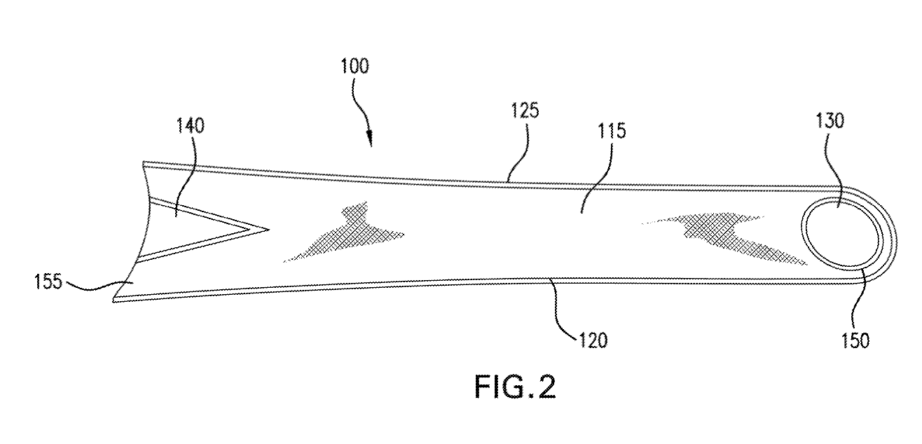

As shown in FIG. 2, the exemplary disposable stethoscope cover 100 can be seen with exemplary dimensions for the open end 155, cutout 140, the location and dimensions of circular 130, as well as additional exemplary dimensional information about the exemplary disposable stethoscope cover 100. It should be understood that other dimensions can be used, and are certainly within the scope of the exemplary embodiments of the present disclosure.

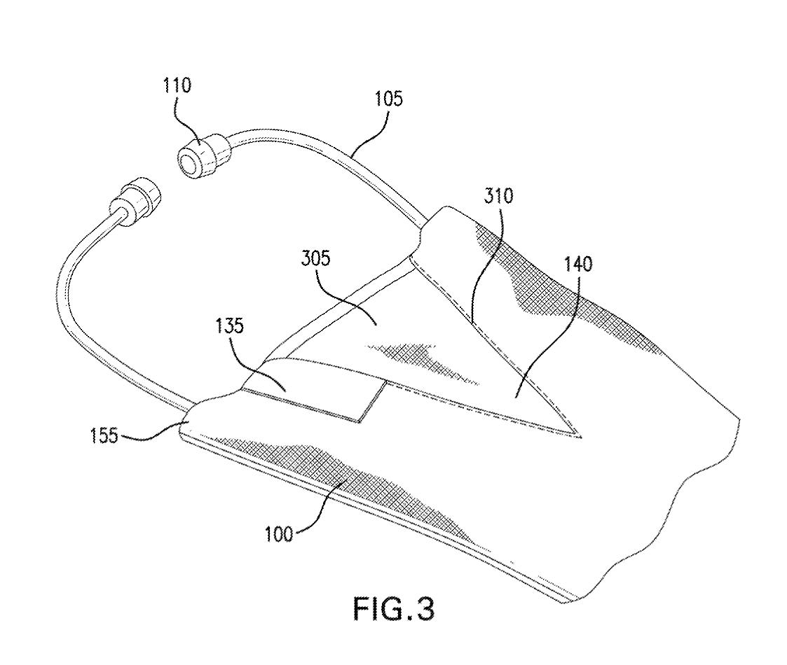

As shown in FIG. 3, an enlarged view of open end 155 is illustrated. The exemplary disposable stethoscope cover 100 can have cutout 140. As illustrated in FIG. 3, cutout 140 can be a triangular shape or a V-shape. However, it should be understood that cutout 140 could be of any shape or design. Cutout 140 can be cutout of both the bottom layer 120 and the top layer 125, forming a pass-through between bottom later 120 and top layer 125. The exemplary disposable stethoscope cover 100 can have two separate elastic pieces 305 woven into the cutout created in bottom layer 120 and in the cutout created in the top layer 125, such that elastic pieces 305 form a continuity in pouch 115 at open end 155. Elastic pieces 305 can be made of a material that provides for elasticity or stretchability. The elasticity of elastic pieces 305 can facilitate open end 155 to be stretched to fit stethoscope 105 around the user's head when using stethoscope 105. Elastic pieces 305 can be individually stitched together 310 with pouch 115, or elastic pieces 305 can be individually attached to pouch 115 using any know method of joining two pieces of fabric or material.

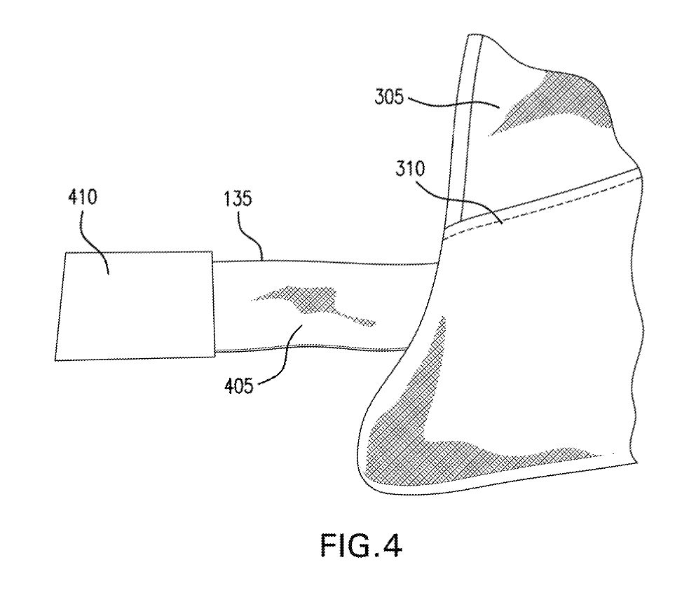

As shown in FIG. 4, the exemplary disposable stethoscope cover 100 can have a strap 135 that can be used to secure and/or hold stethoscope 105 inside of pouch 115. Strap 135 can have an elongated piece 405, which can be made of the same or different material than that of pouch 115. Strap 135 can also have a closure piece 410. Closure piece 410 can have a semi-permanent adhesive, which can be covered with a wax strip. Alternatively, closure piece 410 can be made of a hook and loop type system such that closure piece 410 can be removable attached to pouch 115.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, e.g., data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

What is claimed is:

1. A stethoscope cover comprising:
an enclosure having a closed end and an open end;
a first arrangement located substantially near the closed end and configured to hold a bell of a stethoscope;
an elastic second arrangement at the open end and configured to extend the enclosure when earpieces of the stethoscope are pulled apart.

2. The stethoscope cover of claim 1, wherein the elastic arrangement is substantially v-shaped.

3. The stethoscope cover of claim 1, further comprising a strap configured to hold the stethoscope in the enclosure.

4. The stethoscope cover of claim 3, wherein the stethoscope cover is made from a first material and the strap is made from a second material that is different from the first material.

5. The stethoscope cover of claim 3, wherein the strap includes a closure portion having a semi-permanent adhesive to attach one end of the strap to the stethoscope cover.

6. The stethoscope cover of claim 3, wherein the strap includes a closure portion including a hook and loop system to attach one end of the strap to the stethoscope cover.

7. The stethoscope cover of claim 1, wherein the first arrangement includes a circular element.

8. The stethoscope cover of claim 1, wherein the first arrangement contains an antimicrobial agent.

9. The stethoscope cover of claim 1, wherein the stethoscope cover is made from a first material and the first arrangement is made from a second material that is different from the first material.

10. The stethoscope cover of claim 1, wherein the first arrangement is made from a non-porous material.

11. The stethoscope cover of claim 1, wherein the first arrangement extends a particular distance away from a surface of the stethoscope cover.

12. The stethoscope cover of claim 1, wherein the first arrangement comprises at least two discs layered one on top of the other.

13. The stethoscope cover of claim 12, wherein the first arrangement contains at least one of an anti-microbial agent or a drug.

14. The stethoscope cover of claim 1, wherein the stethoscope cover is sized to substantially cover an entirety of the stethoscope.

15. The stethoscope cover of claim 1, wherein the stethoscope cover is sized to substantially cover an entirety of the stethoscope except for earpieces of the stethoscope.

16. The stethoscope cover of claim 1, wherein the stethoscope cover is of unitary construction.

17. The stethoscope cover of claim 1, wherein the stethoscope cover is constructed from a top layer stitched to a bottom layer.

18. The stethoscope cover of claim 17, wherein the first arrangement is located on the top layer.

19. The stethoscope cover of claim 17, wherein the elastic second arrangement comprises two elastic portions, one elastic portion on the top layer and one elastic portion on the bottom layer.
